(19) United States
(12) Patent Application Publication
(10) Pub. No.: US 2015/0316524 A1
(43) Pub. Date: Nov. 5, 2015

(54) System and Apparatus for a Laboratory Scale Reactor

(71) Applicant: Clean Diesel Technologies, Inc., Oxnard, CA (US)

(72) Inventor: Randal L. Hatfield, Port Hueneme, CA (US)

(21) Appl. No.: 14/800,216

(22) Filed: Jul. 15, 2015

Related U.S. Application Data
(63) Continuation of application No. 13/891,758, filed on May 10, 2013, now abandoned.
Application No. 13/891,758 is a continuation of application No. 13/891,773, filed on May 10, 2013.
Application No. 13/891,773 is a continuation of application No. 13/891,745, filed on May 10, 2013.

(51) Int. Cl. G01N 33/00

(52) U.S. Cl. CPC G01N 33/0073

(57) ABSTRACT

The present disclosure may include a device for testing catalysts, and a method for controlling the flow rate and temperature parameters during the process. The device may separate mass flow control through heating elements from the mass flow through the sample, as well as separate banks for mixing oxidizing elements, carbon dioxide, and diluent gas as well as reducing agents, nitric oxide, and diluent gas. The device disclosed here may also use mass control units of a sufficiently high speed so as to allow the desired testing conditions.

### CROSS-REFERENCE TO RELATED APPLICATIONS

This application is a continuation of U.S. Ser. No. 13/891,758, U.S. Ser. No. 13/891,773, and U.S. Ser. No. 13/891,745, each filed on May 10, 2013 and incorporated herein by reference as if set forth in their entireties.

### BACKGROUND

1. Field of the Disclosure

The present disclosure relates to a laboratory test device and, more particularly, to a device for testing catalysts under dynamic conditions.

2. Background Information

Catalysts may need to be tested to evaluate their performance and their response to parameter changes. Devices of use in testing catalysts may include one or more combustion engines; however, the use of these engines may be expensive, require higher maintenance than desired, and be more time consuming. Additionally, the use of these engines may not allow individual parameter variations or calibrations of use when testing catalysts. Other test devices suitable for testing catalysts may include Laboratory Scale Reactors, commonly referred to as Test Benches, and may allow a greater control over the testing conditions of the catalyst.

However, Laboratory-scale reactors may not capture the catalyst's response to dynamic changes in one or more of multiple variables, including temperature, space-velocity, and reactant gas concentration. This may be of great relevance to catalyst applications where the performance of the catalyst may be judged as a sum of its performance in one or more sequence of events, where the events may have varying space velocities, temperatures and gas systems.

As such, there is a continuing need for test devices able to evaluate the performance of catalysts under a variety of dynamic conditions.

### SUMMARY

The present disclosure may include a device for testing catalysts, and a method for controlling the flow rate and temperature parameters during the process.

The method may include isolating the load perceived by the heating elements from the loading perceived by the catalyst being tested, where excess gas may undergo any suitable venting, including venting over a catalyst holder, venting to a confined environment, venting to the general environment, or any suitable combination. This may allow the space-velocity of gas processed by the heater to vary from the space-velocity of the gas flowing through the sample.

The unit that may control the flow of gas through the catalyst sample may include one or more suitable mass controllers, where the mass controllers may be heated above the dew-point that may be associated with the water vapor concentration. Where a plurality of mass controllers may be used, the mass controllers may be placed in parallel. Suitable mass controllers of use in controlling the flow through the heater and controlling the gas composition may be of a suitably high speed, including mass controllers able to change flow from about 10% flow potential to about 90% of flow potential in less than one second. Mass flow controllers of use may include mass controllers able to make the change in 0.1 seconds.

The method may also include using separate banks of mass flow controllers for mixing the gas composition to the desired composition and for controlling the flow of the gas composition through the heater. A separate bank may be used for controlling any suitable mix of reducing agents, nitric oxide, and diluent gas; while another separate bank may be used for controlling any suitable mix of oxidizing gases, carbon dioxide, and diluent gas. The flow of gas through each bank may be controlled so as to result in any suitable gas composition, including embodiments where the amount of gas flowing through each bank may be controlled to be about half of the flow, where the amount of gas flowing through each bank may be regulated by regulating the amount of diluent gas flowing through each bank. Embodiments where each of the banks may contribute about half of the flow may allow the events that may be generated in each of the banks to reach the catalyst sample at about the same time.

Numerous other aspects, features and advantages of the present disclosure may be made apparent from the following detailed description, taken together with the drawing figures.

### BRIEF DESCRIPTION OF THE DRAWINGS

These and further features, aspects and advantages of the embodiments of the present disclosure will be apparent with regard to the following description, appended claims and accompanying drawings where:

FIG. 1 illustrates a flow chart for the testing process in a test bench reactor.

FIG. 2 illustrates a Gas Feed System.

FIG. 3 illustrates a Test Gas Generator.

FIG. 4 illustrates a Sample Tester.

FIG. 5 illustrates a Test Bench.

It should be understood that these drawings are not necessarily to scale and they can illustrate a simplified representation of the features of the embodiments of the disclosure.

### DETAILED DESCRIPTION

### Definitions

As used here, the following terms have the following definitions:

Mass flow controller (MFC) refers to any computer controlled analog or digital device of use in controlling the flow rate of fluids and/or gases.

Temperature controller refers to any device of use in controlling temperature in a process.

Laboratory Scale Reactor/Test Bench refers to any apparatus suitable for testing a material with a test gas.

Oxidizing agent refers to any substance that may take electrons from another substance in a redox chemical reaction.

Reducing agents refers to any substance that may give electrons to another substance in a redox chemical reaction.

Gas mixture refers to the mixture obtained from combining oxidizing agents, reducing agents, inert gases, or any other suitable gases.

Water-gas mixture refers to the mixture obtained from combining water vapor with a gas mixture.

Test Gas refers to any gas mixture of use in chemically testing an interaction between it and one or more materials.

Catalyst refers to one or more materials that may be of use in the conversion of one or more other materials.

### DESCRIPTION

The description of the drawings, as follows, illustrates the general principles of the present disclosure with reference to various alternatives and embodiments. The present disclosure may, however, be embodied in different forms and should not be limited to the embodiments here referred. Suitable embodiments for other applications will be apparent to those skilled in the art.

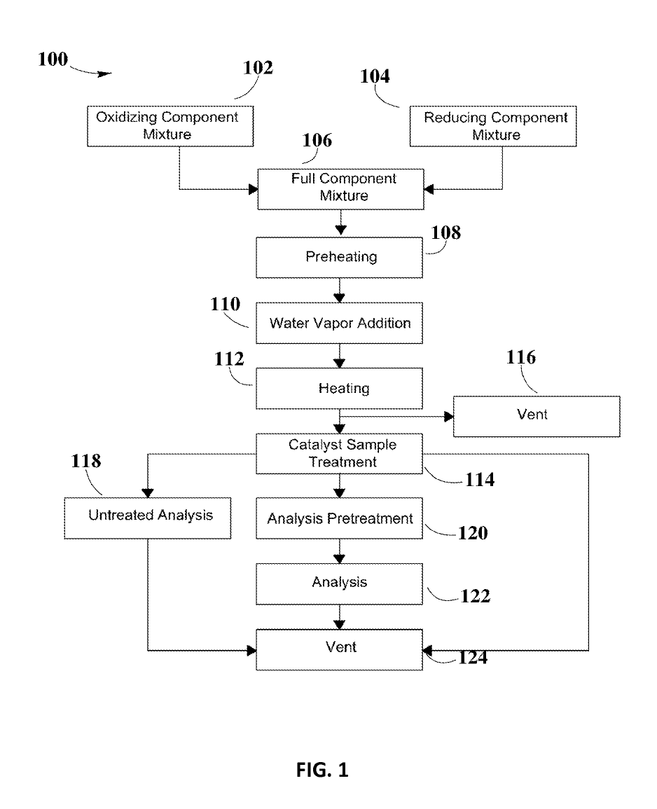

FIG. 1 is a flowchart for a method of testing a material in a Laboratory Scale Reactor. Testing Process 100 may include the preparation of Oxidizing Component Mixture 102 and may include the preparation of Reducing Component Mixture 104. Oxidizing Component Mixture 102 and Reducing Component Mixture 104 may then be mixed and may form Full Component Mixture 106, which may then undergo Preheating 108. Full Component Mixture 106 may then undergo Water Vapor Addition 110, where Full Component Mixture 106 may then undergo Heating 112. A portion of Full Component Mixture 106 may then undergo Catalyst Sample Treatment 114, where any portion not undergoing Catalyst Sample Treatment 114 may undergo venting in Vent 116. A portion of Full Component Mixture 106 having undergone Catalyst Sample Treatment 114 may then be analyzed in any suitable Untreated Analysis 118. Another portion may undergo Analysis Pretreatment 120 previous to undergoing Analysis 122. Any portion not undergoing analysis may be vented in Vent 124, as well as any portion having already undergone Untreated Analysis 118 or Analysis 122.

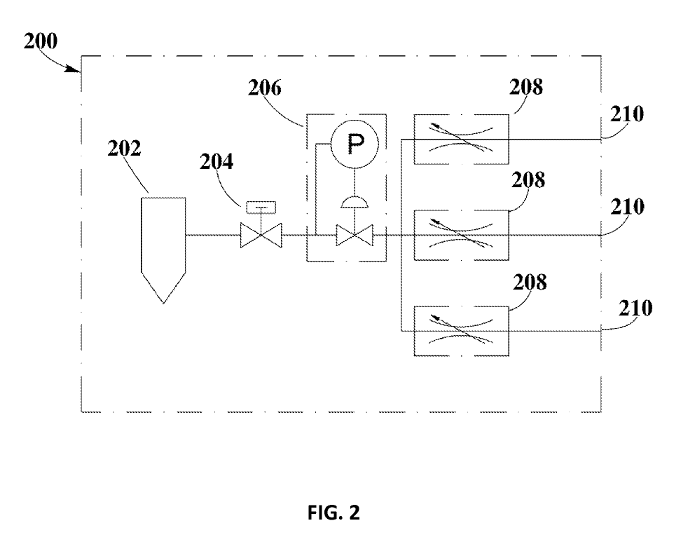

FIG. 2 shows Gas Feed System 200. Gas Feed System 200 may include Gas Source 202, Control Valve 204, Pressure Regulator 206, one or more Mass Flow Controllers 208, and one or more Output Lines 210.

Gas Source 202 may be any source suitable for delivering any suitable gas to the system, including any tank or line able to provide N2, C3H6, C3H8, H2, CO, NO, NO2, CO2, SO2 or any suitable combination thereof at any suitable concentration.

Control Valve 204 may be any valve suitable for restricting or allowing flow from Gas Source 202, including solenoid valves, hydraulic valves, pneumatic valves, or any suitable combination.

Pressure Regulator 206 may be any device suitable for regulating the pressure of gas in Gas Feed System 200, including devices including any suitable pressure gauge or pressure transducer as well as any suitable valve, including solenoid valves, hydraulic valves, pneumatic valves, or any suitable combination.

Mass Flow Controllers 208 may be any mass controller or series of mass controllers suitable for controlling the flow of gas from Gas Source 202 to one or more Output Lines 210 at a suitable frequency, including frequencies in the range of 1 to 25 Hz. Suitable Mass Flow Controllers 208 may include mass flow controllers able to provide any suitable flow rate, including flow rates between 100 cubic centimeters per minute to 60000 cubic centimeters.

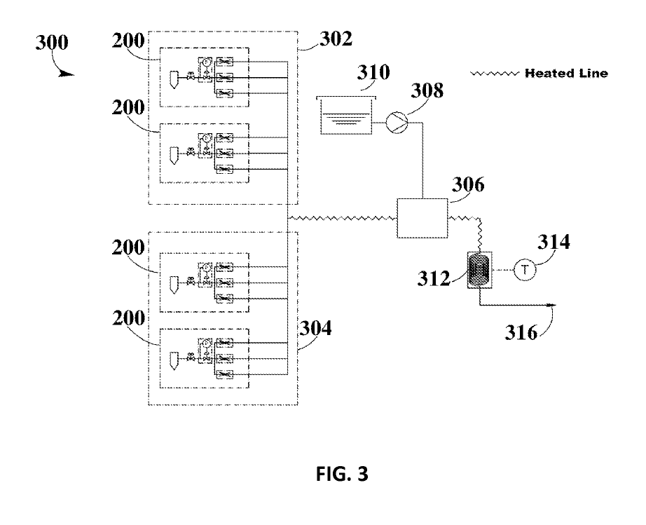

FIG. 3 shows Test Gas Generator 300, having Oxidizing Components Branch 302, Reducing Components Branch 304, Evaporation Block 306, Pump 308, Water Reservoir 310, Heater 312, Temperature Controller 314, and Output 316.

Oxidizing Components Branch 302 may include any number of suitable Gas Feed Systems 200, where the included Gas Feed Systems 200 may provide any number of oxidizing gases, dilutants, and combinations thereof, including N2, O2, and CO2.

Reducing Components Branch 304 may include any number of suitable Gas Feed Systems 200, where the included Gas Feed Systems 200 may provide any number of reducing gases, dilutants, and combinations thereof, including N2, H2, CO, NO, and any suitable hydrocarbons. Suitable Hydrocarbons may include C3H8. Suitable heavy hydrocarbons may also be added using any suitable method, including liquid injection and evaporation. Suitable heavy hydrocarbons may include decane, tolune, and dodecane.

The flow of the mixture of gases generated by Oxidizing Components Branch 302 and Reducing Components Branch 304 may then be preheated by any suitable means, including heated lines, where the heated lines may be heated using heat jackets. Suitable temperatures may include temperatures in the range of 130° C. to 180° C., including 150° C.

Evaporation Block 306 may be any device suitable for evaporating water and adding it to the flow of gas generated by the combination of gas flows from Oxidizing Components Branch 302 and Reducing Components Branch 304 in Test Gas Generator 300. Evaporation Block 306 may evaporate water which may be provided by Pump 308, where Pump 308 may be any pump suitable for pumping water from Water Reservoir 310 to Evaporation Block 306. Suitable temperatures in Evaporation Block 306 may include temperatures in the range of 110° C. to 150° C., including 130° C.

The gas flowing out of Evaporation Block 306 may then be heated by Heater 312, where Heater 312 may be any suitable heating device, including serpentine heaters. Heater 312 may be controlled by Temperature Controller 314, which may be any suitable temperature controller, including thermocouples and thermistors.

The resulting test gas exits Test Gas Generator 300 through Output 316.

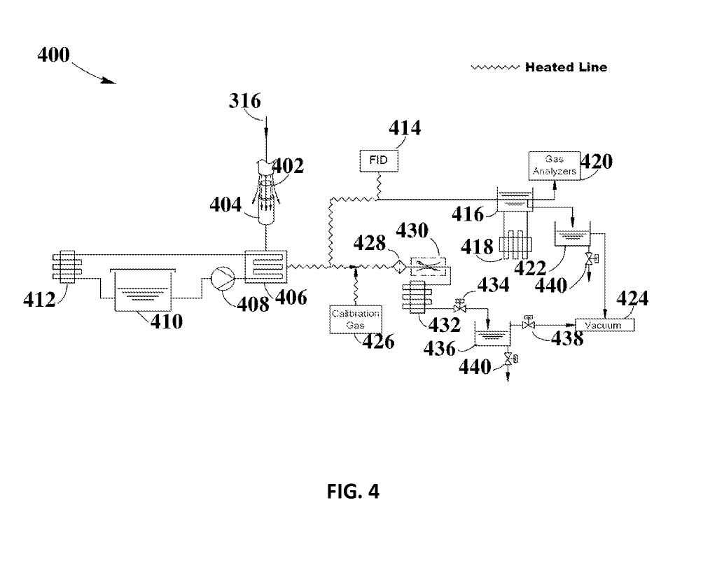

FIG. 4 shows Sample Tester 400, including Catalyst Sample 402 on Catalyst Holder 404, Heated Block 406, Pump 408, Cooling Liquid Reservoir 410, Radiator 412, FID Unit 414, Cooling Bath 416, Chiller Unit 418, Gas Analyzer 420, Water Reservoir 422, Vacuum 424, Calibration Gas 426, Filter 428, Heated Mass Flow Controller 430, Radiator 432, Control Valve 434, Water Reservoir 436, Control Valve 438, and Purge Valves 440.

Catalyst Sample 402 may be any material suitable for testing with test gas delivered by Output 316, placed on any suitable Catalyst Holder 404. Catalyst Sample 402 may interact with any suitable portion of test gas delivered by Output 316, where any portion not of test gas delivered by Output 316 may undergo any suitable venting, including venting through Catalyst Holder 404 and venting to the environment.

The temperature of test gas treated by Catalyst Sample 402 may then be controlled by Heated Block 406, where Heated Block 406 uses cooling liquid provided by Pump 408 from Cooling Liquid Reservoir 410. Cooling liquid in Cooling Liquid Reservoir 410 may be any suitable cooling liquid, including water, ethylene glycol, propylene glycol, or any suitable combination thereof. Cooling liquid exiting Heated Block 406 may then be cooled by Radiator 412.

A suitable portion of test gas exiting Heated Block 406 may then flow through heated lines to FID Unit 414, where FID unit 414 may be any suitable Flame Ionization Detector device.

Another suitable portion of test gas exiting Heated Block 406 may be cooled to a suitable temperate in Cooling Bath 416. Cooling Bath 416 allows the test gas to be cooled to a temperature suitable for condensing the water vapor content in the incoming test gas, and is kept at a suitable temperature using Chiller Unit 418, where Chiller Unit 418 may be any suitable chilling device. Dry test gas exiting Cooling Bath 416 may then be analyzed by one or more suitable Gas Analyzers 420. Moisture condensed in Cooling Bath 416 may flow into Water Reservoir 422, where the moisture may then exit to Vacuum 424 or be purged by Purge Valve 440.

Another suitable portion of test gas exiting Heated Block 406 may then flow through one or more suitable Filters 428. The flow of gas may be controlled by one or more suitable Heated Mass Flow Controllers 430, where Heated Mass Flow Controllers 430 may provide a suitable flow rate, including rates between 0 to 100 liters per minute. Test gas flowing through Heated Mass Flow Controllers 430 may then be cooled in Radiator 432, where it may then flow through control Valve 434. Control Valve 434 may be any valve suitable for restricting or allowing flow from Heated Mass Flow Controllers 430, including solenoid valves, hydraulic valves, pneumatic valves, or any suitable combination.

During calibration of one or more of FID Unit 414 and/or Gas Analyzers 420, Heated Mass Flow Controllers 430 may be set to a suitably low flow value, including zero. Calibration Gas 426 may then flow to FID Unit 414 and through Cooling Bath 416 to Gas Analyzers 420, and may also flow through Catalyst Sample 402 in a direction which may be contrary to that of flow in normal operating conditions.

Test gas exiting Control Valve 434 may then flow into Water Reservoir 436, where it may then flow through Control Valve 438 into Vacuum 424, or may be purged intermittently along with the water when Water Reservoir 436 is emptied.

Control Valve 438 may be any valve suitable for restricting or allowing flow from Water Reservoir 436, including solenoid valves, hydraulic valves, pneumatic valves, or any suitable combination.

One or more Purge Valves 440 may be used to purge Water Reservoir 422 and/or Water Reservoir 436, where suitable valves may include solenoid valves, hydraulic valves, pneumatic valves, manually activated valves, or any suitable combination.

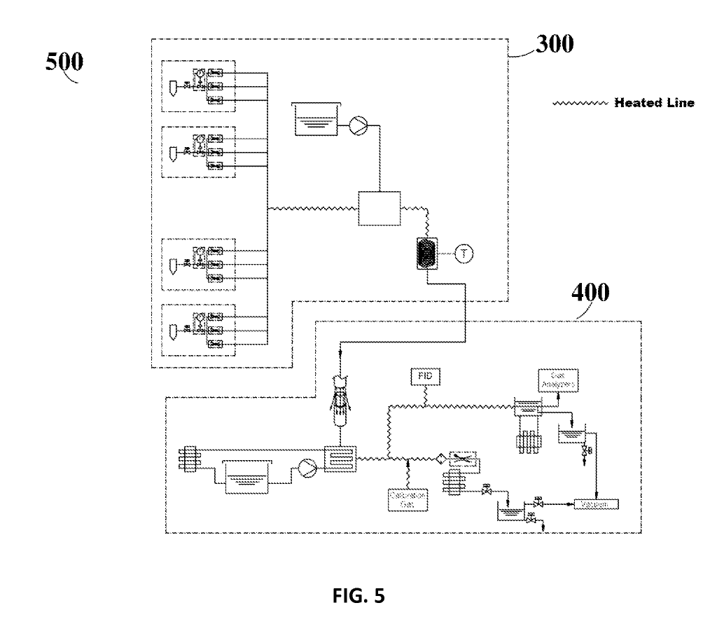

FIG. 5 show Test Bench 500, including Test Gas Generator 300 and Sample Tester 400.

What is claimed is:

1. An apparatus for the preparation of gas mixtures, comprising:
at least two gas delivery banks, each for delivering at least one of a plurality of gasses, and each comprising:
at least one gas source for receiving at least one input gas;
at least one control valve operable on the at least one input gas;
at least one pressure regulator operable on the at least one input gas;
a plurality of first mass flow controllers operable on the at least one input gas; and
at least one output for outputting a mass-flow controlled, regulated one of the at least one input gas as the delivered at least one of the plurality of gasses;
at least one evaporation block suitable for adding H₂O to a mixture of more than one of the delivered at least one of the plurality of gasses received from a respective one of the at least two gas delivery banks;
wherein the ratio of the mixture of the more than one of the delivered at least one of the plurality of gasses is at least partially controlled by respective ones of the plurality of first mass flow controllers of ones of the at least two gas delivery banks;
a heating chamber comprising at least one heating element and a heating controller suitable for heating the mixture to a controlled heating temperature and for imparting a first space velocity to the mixture;
at least one catalyst sample provided substantially in-line following the heating chamber and suitable for interacting with a first portion of the mixture having a second space velocity;
at least one vent suitable for venting, prior to interacting with the catalyst sample, of a second portion of the mixture, thereby imparting the second space velocity to the first portion of the mixture;
at least one second mass flow controller for controlling flow of a mass-flow controlled one of the interacted first portion of the mixture; and
at least one gas analyzer suitable for analyzing the mass-flow controlled one of the interacted first portion of the mixture.

2. The apparatus of claim 1, further comprising at least one calibration source providing at least one calibration gas wherein the at least one second mass flow controller effects the interaction of the at least one calibration gas with the interacted first portion of the mixture.

3. The apparatus of claim 1, wherein the evaporation block may have a temperature of about 110° C. to about 150° C.

4. The apparatus of claim 1, wherein the evaporation block may have a temperature of about 130° C.

5. The apparatus of claim 1, wherein at least one of the plurality of gasses comprises at least one oxidizing component.

6. The apparatus of claim 5, wherein the at least one oxidizing component comprises at least one selected form the group consisting of N₂, H₂, O₂, CO₂, and combinations thereof.

7. The apparatus of claim 1, wherein at least one of the plurality of gasses comprises at least one reducing component.

8. The apparatus of claim 7, wherein the at least one reducing component comprises at least one selected form the group consisting of N₂, H₂, CO, NO, C3H₈, C₁₀H₂₂, C₇H₈, CH₃(CH₂)₁₀CH₃ and combinations thereof.

9. The apparatus of claim 1, wherein at least one of the plurality of gasses comprises at least one one diluent gas.

10. The apparatus of claim 1, further comprising at least one control valve, wherein the at least one control valve is selected from the group consisting of a solenoid valve, a hydraulic valve, a pneumatic valve, and a combination thereof.

11. The apparatus of claim 1, wherein the heating may be from about 130° C. to about 180° C.

12. The apparatus of claim 1, wherein the heating may be to about 150° C.

13. The apparatus of claim 1, wherein the gas analyzer is a flame ionization detector.

14. The apparatus of claim 1, wherein the at least one second mass flow controller provides a flow rate of about 0 to about 100 liters per minute.

15. The apparatus of claim 2, wherein the at least one second mass flow controller provides a flow rate of about 0 liters per minute and wherein the at least one calibration gas flows to the at least one gas analyzer.

16. The apparatus of claim 1, further comprising a plurality of condensors and at least one vaccum source, wherein the vacuum source allows liquids condensed from the mixture of the at least one second gas and the at least one third gas to be purged from the at least one apparatus for analyzing a fluid.

17. The apparatus of claim 1, wherein the first space velocity is not equal to the second space velocity.
